(19) United States
(12) Patent Application Publication
(10) Pub. No.: US 2008/0181937 A1
(43) Pub. Date: Jul. 31, 2008

(54) DOSAGE REGIMEN AND MEDICAMENT FOR GUARDING MEMORY AND BRAIN HEALTH AND FOR PREVENTING OR REDUCING RISK OF ONSET OF DEMENTIA BY ADMINISTRATION OF SPECIFIC VITAMINS AND SUPPLEMENTS (MEMOGUARD)

(76) Inventor: Majid Fotuhi, Cockeysville, MD (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/021,002

(22) Filed: Jan. 28, 2008

Related U.S. Application Data
(60) Provisional application No. 60/887,101, filed on Jan. 29, 2007.

(51) Int. Cl. A61K 9/70; A61K 31/355; A61P 25/28

(52) U.S. Cl. 424/449; 514/458

(57) ABSTRACT

Methods and compositions for preventing memory loss and reducing the onset of Alzheimer's disease and the pathological symptoms associated therewith including memory loss, reduced cognitive function and dementia are provided. These methods and compositions comprise dosage regimens that provide for the daily administration of effective amounts of Vitamin C, Vitamin E, DHA, and Curcumin.

### RELATED APPLICATIONS

This application claims priority to U.S. provisional Ser. No. 60/887,101 filed on Jan. 29, 2007. This application is incorporated by reference in its entirety herein.

### BACKGROUND OF THE INVENTION

With the current increase in the numbers and proportion of the elderly in the population, cognitive decline and dementia are major public health issues. Alzheimer's disease, the most common form of dementia, already challenges our health care system in the United States and Canada, and the associated costs are projected to skyrocket in the coming decades. Disabling memory loss is not limited to Western countries. Roughly, 24.3 million people globally have dementia, and 4.6 million cases arise every year. There is a new case of dementia arising every seven seconds. Given these numbers, there is an urgent need to find interventions that can effectively slow or even prevent the onset of dementia and the rate of cognitive decline associated with aging.

Currently, the underlying causes of dementia are not entirely understood. It is known that inflammation and the production of reactive oxygen apparently play a major role in the neuronal injury and brain atrophy that occur during aging and dementia. It is also known that during the early stages of Alzheimer's disease that a specific protein (amyloid) accumulates in the brain and forms gum-like insoluble plaques (amyloid plaques). These plaques are known to damage brain cells directly and indirectly by triggering inflammatory reactions, which in turn cause more brain damage.

Inflammation is a Main Culprit in Aging and Alzheimer's

There is compelling evidence that inflammation is a key process contributing to brain damage that happens with aging and particularly with Alzheimer's dementia. For example, inflammatory markers are elevated in patients exhibiting dementia. However, to date there is no medication available to specifically target inflammatory processes and help to prevent dementia.

There is some anecdotal evidence that mental health is better maintained in individuals who conduct daily activities that are stimulating to the brain. For example an autopsy study of the brains of nuns who were very mentally active until death (many of whom had lived until their late 80's and 90's) revealed that a substantial number of these autopsied brains appeared to exhibit significant anatomical signs of plaques and disease. Notwithstanding, these individuals did not display any clinical symptoms of dementia or Alzheimer's at the time of their death. Based on this and other anecdotal evidence, the Alzheimer's Association recently began a publicity campaign to promote healthy eating, exercise, and brain stimulation in order to maintain brain function during aging. For example, they have promoted elderly people to conduct daily mental exercises such as completing mazes or crossword puzzles or playing computer games to maintain mental agility. However, they have not yet offered any specific means or medicament for preventing dementia and Alzheimer's disease.

Several new medications are helpful in reducing the severity and frequency of symptoms in patients with Alzheimer's disease. These include Aricept, Razadyne, Exelon, and Namenda. The first two of these medications have been studied in patients with marked memory loss at risk for developing Alzheimer's disease. Neither of these drugs were shown to significantly prevent the onset of Alzheimer's disease. This is not surprising; one of these drugs target the inflammation that appears to be a main contributor of brain damage in dementia.

### BRIEF DESCRIPTION AND OBJECTS OF THE INVENTION

It is an object of the invention to provide a method of treating an individual who is at increased risk of developing Alzheimer's disease because of family history, one or more genetic frisk actors associated with an increased risk of developing Alzheimer's disease, or environmental factors correlated to an increased risk of developing Alzheimer's disease, comprising treating said individual with a treatment regimen that provides for the oral administration or transdermal absorption of a composition containing Vitamin C, Vitamin E, DHA and Curcumin.

It is a more specific object of the invention to provide such treatment methods wherein the amounts of these constituents that are administered or absorbed once daily are (i) an amount of vitamin C will vary from about 40 mg to about 4000 mg daily, (ii) an amount of vitamin E ranging from about 20 IU to about 2000 iu daily; (iii) an amount of Curcumin ranging from about 20 mg to about 2000 mg daily, and (iv) an amount of DHA ranging from about 40 mg to about 4000 mg daily.

It is an even more specific object of the invention to provide such treatment methods wherein the amounts of the 4 constituents are: (i) an amount of vitamin C ranging from 80 to 200 mg daily, (ii) an amount of vitamin E ranging from about 50 iu to about 1000 iu daily, (iii) an amount of Curcumin ranging from about 40 to about 1000 mg daily and (iv) an amount of DHA ranging from about 80 to 2000 mg daily.

It is an even most specific object of the invention to provide such treatment methods wherein the amounts of said 4 constituents are: (i) an amount of vitamin C ranging from about 200 to 600 mg daily; (ii) an amount of vitamin E ranging from about 100 to about 300 iu daily; (iii) an amount of Curcumin ranging from about 50 mg to about 400 mg daily; and (iv) an amount of DHA 1 ranging from about 200 to 600 mg daily.

It is an especially preferred object of the invention to provide such a treatment method wherein the administration regimen provides for the daily ingestion or transdermal delivery of about (i) 400 mg vitamin C, (ii) about 200 iu of vitamin E, (iii) about 200 mg of Curcumin and (iv) about 400 mg of DHA.

It is also a preferred object of the invention to provide for such treatment methods to be effected using a tablet, capsule or transdermal composition.

It is also an object of the invention to provide a medicament for reducing or slowing the onset of the pathological symptoms associated with Alzheimer's disease in an individual at increased risk of developing Alzheimer's disease which contains an effective amount of at least 4 constituents: Vitamin E, Vitamin C, Curcumin and DHA.

It is a more specific object to provide such a medicament wherein the effective amounts of these 4 constituents contained therein on daily administration or which are transdermally absorbed daily on administration are (i) an amount of vitamin C will vary from about 40 mg to about 4000 mg, (ii) an amount of vitamin E ranging from about 20 IU to about 2000 iu; (iii) an amount of Curcumin ranging from about 20 mg to about 2000 mg daily, and (iv) an amount of DHA ranging from about 40 mg to about 4000 mg daily.

It is an even more specific object of the invention to provide such a medicament wherein the effective amounts of the 4 constituents contained therein on daily administration or transdermally absorbed daily are: (i) an amount of vitamin C ranging from 80 to 200 mg, (ii) an amount of vitamin E ranging from about 50 iu to about 1000 iu, (iii) an amount of Curcumin ranging from about 40 to about 1000 mg and (iv) an amount of DRA ranging from about 80 to 200 mg.

It is an even more specific object of the invention to provide such a medicament wherein the effective amounts of said 4 constituents contained therein on daily administration or transdermally absorbed on administration are: (i) an amount of vitamin C ranging from about 200 to 600 mg daily; (ii) an amount of vitamin E ranging from about 100 to about 300 iu daily; (iii) an amount of Curcumin ranging from about 50 mg to about 400 mg daily; and (iv) an amount of DHA 1 ranging from about 200 to 600 mg daily.

It is an especially preferred object of the invention to provide such a medicament wherein the medicament provides for the daily ingestion or transdermal delivery of about (i) 400 mg vitamin C, (ii) about 200 iu of vitamin E, (iii) about 200 mg of Curcumin and (iv) about 400 mg of DHA.

In the preferred object of the invention such medicaments will comprise tablet, capsule or transdermal compositions.

### DETAILED DESCRIPTION OF THE INVENTION

As noted above, serum inflammatory markers are elevated in patients with dementia. Some observational studies have shown that individuals taking non-steroidal anti-inflammatory drugs (NSAID's) such as ibuprofen exhibit a lower incidence of Alzheimer's disease. Other studies have shown improved cognition in elderly patients taking the anti-oxidant vitamins E and C. Vitamin E levels are sometimes low in patients with cognitive impairment or dementia and treatment thereof with vitamin E can even delay transfer to a nursing home. These studies favor use of NSAID's and/or anti-oxidant vitamins for preventing dementia.

Because the reported research into the cause of brain damage in Alzheimer's disease points to inflammation, anti-inflammatory medications have been suggested as a preventative. For example, patients who suffer from rheumatoid arthritis and take anti-inflammatory drugs on a daily basis have a reduced incidence of Alzheimer's disease onset. Other studies, including one made at Johns Hopkins, revealed that participants taking vitamins E and C were less likely to become demented. However, to the best of the inventors' knowledge no study has suggested the combined administration of NSAID's and anti-oxidant vitamins E and C.

In a multi-center epidemiological study involving 5000 participants who were monitored for eight years, Dr. Fotuhi and his colleagues at Johns Hopkins recently showed that NSAID's combined with vitamin E and C is associated with less cognitive decline. However, the benefits of NSAID's such as Motrin need to be balanced with its potential side effects including ulcers and heart disease. As such, inventor has decided to substitute two natural anti-inflammatory drugs instead of traditional NSAID's. The two natural anti-inflammatory agents he has chosen are DHA and curcumin.

DHA: One of the exciting new discoveries in the field of Alzheimer's prevention has been the realization that the omega-3 fatty acid, DHA, can reduce the amount of amyloid in the brain. Alzheimer's patients, and in fact even those with depression or learning disability, have low levels of DHA. Adding DHA to the diet in the form of eating fish 2-3 times a week or taking supplements increases memory and attention in normal healthy subjects and significantly lowers the risk of Alzheimer's in the elderly. DHA is safe. American Heart Association recommends it for patients with coronary heart disease. Infant formula preparations contain DHA to help with brain development. Small clinical trials in Japan and Sweden have shown positive results and large scale placebo-controlled trials are now underway in the United States.

Curcumin: The brain protective properties of Curcumin, the active ingredient in the curry spice, have been surprising. Indians, who have 75% less risk of developing Alzheimer's than Americans, consume curry on a regular basis. They also rub it on the skin and joints to heal wounds and reduce pain. In fact, in a recent study in the University of Kansas, Dr. Barbara Timmermann and her colleagues discovered that Curcumin can reduce inflammation in an animal model of rheumatoid arthritis. Curcumin is a “natural” NSAID without all the GI side effects and has been shown to be fairly safe up doses of 8000 mg in clinical trials for its use for treatment of cancer.

Therefore, based on the foregoing, and based on other observations of the inventor, the present invention provides novel and improved methods and medicament compositions for use therein for the preventing and/or slowing of the onset of the pathological symptoms associated with Alzheimer's disease in patients at risk of developing Alzheimer's diseases because of a family history or other factors such as environmental factors, genetic markers, and the like. In addition the invention provides methods for reducing the onset of the pathological symptoms associated with Alzheimer's disease in patients already exhibiting symptoms associated with the early or latter stages of Alzheimer's disease such as memory loss, cognitive decline, and dementia.

The subject prevention and treatment methods will comprise the daily administration or delivery of one or more compositions which together comprise at least four specific constituents, i.e., vitamin E, Vitamin C, DHA and Curcumin and/or pharmaceutically acceptable salts or derivatives thereof. Most preferably these constituents will be contained in the same composition which is administered once daily or in a transdermal formulation which provides for the release of effective amounts of these 4 constituents daily.

This composition will preferably be suitable for oral administration and may be in the form of a tablet, capsule, pellet, or other orally administrable form

Alternatively, this composition may be in the form of a transdermal formulation, e.g. a transdermal patch which is designed to release a sufficient amount of each of the four constituents daily to provide for a reduction or slowing in the onset of the pathological symptoms associated with Alzheimer's disease, especially reduced cognition capabilities and memory loss in a patient at risk of developing or exhibiting signs of Alzheimer's disease.

The relative amounts of these constituents may be varied. In general the amount of vitamin C administered daily will vary from about 40 mg to about 4000 mg daily, more preferably will range from about 80 to 200 mg daily, and most preferably will range from about 200 to 600 mg.

The amount of vitamin E administered daily in the subject methods will typically range from about 20 IU to about 2000 iu daily, more preferably for about 50 iu to about 1000 iu and most preferably from about 100 to about 300 iu.

The amount of Curcumin administered daily will typically range from about 20 mg to about 2000 mg daily, more preferably from about 40 to about 1000 mg daily and most preferably from about 50 mg to about 400 mg.

The amount of DHA administered daily will typically range from about 40 mg to about 4000 mg daily, more preferably will range from about 80 to 200 mg daily, and most preferably will range from about 200 to 600 mg.

An exemplary composition for use in the present invention may comprise a tablet or capsule formulation comprising about 400 mg vitamin C, about 200 iu of vitamin E, about 200 mg of Curcumin and about 400 mg of DHA.

Alternatively, an exemplary formulation for use in the subject methods may comprise a transdermal patch which provides for the daily release and absorption of about 400 mg of vitamin C, about 200 iu if vitamin E, about 200 mg or Curcumin and about 400 mg of DHA.

As mentioned, the subject medicament compositions will be administered to persons at elevated risk of developing Alzheimer's disease, e.g., because of family history, genetic markers which correlate to increased risk of Alzheimer's disease, environmental risk factors that correlate to an increased risk of Alzheimer's disease and the like. For example a person at elevated risk may have a parent who was diagnosed with Alzheimer's disease. The treated individuals will be older individuals, i.e., typically at least 55-60 years of age, or more preferably at least 65 to 70 years of age unless the family history reveals on earlier onset of Alzheimer's disease. Alternatively, the subject compositions may be administered to persons exhibiting symptoms associated with the onset of Alzheimer's disease such as memory loss, a decrease in cognitive abilities, signs of dementia and the like.

Administration of the subject compositions in the prescribed dosages and dosage regimens will slow the onset of Alzheimer's disease and/or reduce the increase in the onset of indicators associated with Alzheimer's disease such as memory loss and loss of cognitive capabilities and dementia.

The subject orally administrable medicaments will comprise at least the recited effective amounts of the 4 recited constituents and may further comprise other actives and non-active ingredients such as flavorings, lubricants, tabletting agents, pharmaceutically acceptable carriers, colorants, and the like.

These medicaments preferably may be imprinted with a proprietary name such as “Memoguard” These medicaments will preferably be packaged with a label which directs their prescribed usage such as once daily oral administration.

The subject invention has been fully described. In order to further describe the invention and its intrinsic advantages the following examples are provided.

### EXAMPLES

### Example 1

A group of at least 50 persons each of whom is at least 60 years of age and who are at elevated risk of developing Alzheimer's disease because of family history (father or mother diagnosed with the disease), all of male gender, are instructed to ingest once-daily a tablet formulation according to the invention containing about 400 mg vitamin C, about 200 iu of vitamin E, about 200 mg of Curcumin and about 400 mg of DHA. These persons are also instructed to maintain a log documenting their maintaining this daily dosage regimen. The memory and cognitive function of each of these persons is assessed at the start of the treatment regimen and is reassessed periodically (every 3 months). The status of this group of persons is compared to a control group of 50 persons also at elevated risk of developing Alzheimer's disease because of family history and also each at least 60 years old and of male gender who are instructed to ingest once daily a placebo tablet not containing these 4 constituents. These persons are also instructed to maintain a log documenting their maintaining of this daily dosage regimen. As the treatment regimens proceed for each group if any persons are diagnosed with a life threatening disease they are removed from the treatment regimen. The memory and cognitive capabilities of these persons is also assessed prior to treatment and periodically (at least every 3 months).

This comparison will show that over time that the group of persons treated with the tablet medicament composition according to the invention on average exhibit less memory loss and less of a reduction of cognitive function relative to the control group over the same period of time.

### Example 2

A group of at least 50 persons each of whom is at least 60 years of age and who are at elevated risk of developing Alzheimer's disease because of family history (father or mother diagnosed with the disease), all of female gender, and of otherwise good health, are instructed to ingest once-daily a capsule formulation according to the invention containing about 400 mg vitamin C, about 200 iu of vitamin E, about 200 mg of Curcumin and about 400 mg of DHA. These persons are also instructed to maintain a log documenting their maintenance of this dosage regimen. The memory and cognitive function of each of these persons and their health status is assessed at the start of the treatment regimen and is reassessed periodically (every 3 months). The status of this group of persons is compared to a control group of at least 50 persons also at elevated risk of developing Alzheimer's disease because of family history and also each at least 60 years old and of female gender and otherwise of god health who are instructed to ingest once daily a placebo capsule formulation not containing these 4 constituents. These persons are also instructed to maintain a log documenting their maintaining of this daily dosage regimen. The memory and cognitive capabilities and health status of these persons is also assessed prior to treatment and periodically (at least every 3 months). As the trials proceed if any of the treated persons in either group is diagnosed with a life threatening disease then these persons are removed from the treatment regimen and are not included in the evaluation.

This comparison of these 2 groups will show that over time that the group of persons treated with the medicament composition according to the invention on average exhibit less memory loss and less of a reduction of cognitive function relative to the control group over the same time periods.

### Example 3

A group of at least 50 persons each of whom is at least 60 years of age and who are at elevated risk of developing Alzheimer's disease because of family history (father or mother diagnosed with the disease), all of female gender, and of otherwise good health, are provided a transdermal patch according to the invention that provides for the release and daily absorption of about 400 mg vitamin C, about 200 iu of vitamin E, about 200 mg of Curcumin and about 400 mg of DHA. These persons are instructed to remove this transdermal patch daily and to reapply another patch according to the invention and also to maintain a log documenting their maintenance of the prescribed dosage regimen. The memory and cognitive function of each of these persons and their health status is assessed at the start of the treatment regimen and is reassessed periodically (every 3 months). The status of this group of persons is compared to a control group of at least 50 persons also at elevated risk of developing Alzheimer's disease because of family history and also each at least 60 years old and of female gender and otherwise of god health who are instructed to similarly apply a transdermal patch (placebo) formulation not containing these 4 constituents and to remove and reapply another transdermal patch (placebo) daily. These persons are also instructed to maintain a log documenting their maintaining of this dosage regimen. The memory and cognitive capabilities and health status of these persons is also assessed prior to treatment and periodically (at least every 3 months). As the trials proceed if any of the treated persons in either group is diagnosed with a life threatening disease then these persons are removed from the treatment regimen and are not included in the evaluation.

This comparison of these 2 groups will show that over time that the group of persons treated with the medicament composition according to the invention on average exhibit less memory loss and less of a reduction of cognitive function relative to the control group over the same time periods.

In order to further describe the invention and its intended scope the following claims are provided.

1. A method of treating an individual who is at increased risk of developing Alzheimer's disease because of family history, one or more genetic frisk actors associated with an increased risk of developing Alzheimer's disease, or environmental factors correlated to an increased risk of developing Alzheimer's disease, comprising treating said individual with a treatment regimen that provides for the oral administration or transdermal absorption of a composition containing Vitamin C, Vitamin E, DHA and Curcumin.

2. The method of claim 1 wherein the amounts of these constituents that are administered or absorbed once daily are (i) an amount of vitamin C will vary from about 40 mg to about 4000 mg daily, (ii) an amount of vitamin E ranging from about 20 iu to about 2000 in daily; (iii) an amount of Curcumin ranging from about 20 mg to about 2000 mg daily, and (iv) an amount of DHA ranging from about 40 mg to about 4000 mg daily.

3. The method of claim 1 wherein the amounts of the 4 constituents are: (i) an amount of vitamin C ranging from 80 to 200 mg daily, (ii) an amount of vitamin E ranging from about 50 in to about 1000 iu daily, (iii) an amount of Curcumin ranging from about 40 to about 1000 mg daily and (iv) an amount of DHA ranging from about 80 to 200 mg daily.

4. The method of claim 1 wherein the amounts of said 4 constituents are: (i) an amount of vitamin C ranging from about 200 to 600 mg daily; (ii) an amount of vitamin E ranging from about 100 to about 300 iu daily; (iii) an amount of Curcumin ranging from about 50 mg to about 400 mg daily; and (iv) an amount of DHA 1 ranging from about 200 to 600 mg daily.

5. The method of claim 1 wherein the administration regimen provides for the daily ingestion or transdermal delivery of about (i) 400 mg vitamin C, (ii) about 200 iu of vitamin E, (iii) about 200 mg of Curcumin and (iv) about 400 mg of DHA.

6. The method of claim 1 wherein the administered composition is a tablet formulation.

7. The method of claim 1 wherein the administered composition is a capsule formulation.

8. The method of claim 1 wherein the administered composition is a transdermal patch formulation.

9. The method of claim 1 wherein the treated individual has a family history associated with Alzheimer's disease.

10. The method of claim 1 wherein the treated individual has one or more genetic factors associated with an increased risk of developing Alzheimer's disease.

11. The method of claim 1 wherein the cognitive status of this individual is assessed prior to treatment and periodically as the treatment regimen proceeds.

12. A medicament for reducing or slowing the onset of the pathological symptoms associated with Alzheimer's disease in an individual at increased risk of developing Alzheimer's disease which contains an effective amount of at least 4 constituents: Vitamin E, Vitamin C, Curcumin and DHA.

13. The medicament of claim 12 wherein the effective amounts of these 4 constituents contained therein on daily administration or which are transdermally absorbed daily on administration are (i) an amount of vitamin C will vary from about 40 mg to about 4000 mg, (ii) an amount of vitamin E ranging from about 20 IU to about 2000 iu; (iii) an amount of Curcumin ranging from about 20 mg to about 2000 mg daily, and (iv) an amount of DHA ranging from about 40 mg to about 4000 mg daily.

14. The medicament of claim 12 wherein the effective amounts of the 4 constituents contained therein on daily administration or transdermally absorbed daily are: (i) an amount of vitamin C ranging from 80 to 200 mg, (ii) an amount of vitamin E ranging from about 50 iu to about 1000 iu, (iii) an amount of Curcumin ranging from about 40 to about 1000 mg and (iv) an amount of DHA ranging from about 80 to 200 mg.

15. The medicament of claim 12 wherein the effective amounts of said 4 constituents contained therein on daily administration or transdermally absorbed on administration are: (i) an amount of vitamin C ranging from about 200 to 600 mg daily; (ii) an amount of vitamin E ranging from about 100 to about 300 iu daily; (iii) an amount of Curcumin ranging from about 50 mg to about 400 mg daily; and (iv) an amount of DHA 1 ranging from about 200 to 600 mg daily.

16. The medicament of claim 12 wherein the medicament provides for the daily ingestion or transdermal delivery of about (i) 400 mg vitamin C, (ii) about 200 iu of vitamin E, (iii) about 200 mg of Curcumin and (iv) about 400 mg of DHA.

17. The medicament of claim 12 which is a tablet.

18. The medicament of claim 12 which is a capsule.

19. The medicament of claim 12 which comprises a transdermal administrable composition.

20. The medicament of claim 19 which comprises a transdermal patch.
